(19) United States
(12) Patent Application Publication
(10) Pub. No.: US 2011/0159122 A1
(43) Pub. Date: Jun. 30, 2011

(54) COMBINATION OF EXTRACTS OF VARIOUS PLANTS FOR IMPROVING THE SYMPTOMS OF DEMENTIA DISORDERS

(75) Inventor: Bruno Frank, Kleinrinderfeld (DE)

(73) Assignee: Kneipp-Werke Kneipp-Mittel-Zentrale GmbH & Co,KG, Wurzburg (DE)

(21) Appl. No.: 13/061,815

(22) PCT Filed: Aug. 11, 2009
(86) PCT No.: PCT/EP09/60358

§ 371 Date: Mar. 2, 2011

(30) Foreign Application Priority Data
Sep. 5, 2008 (EP) 08015700.1

(51) Int. Cl. A61K 36/254; A61K 36/82; A61K 36/906; A61P 25/28

(52) U.S. Cl. 424/728; 424/729; 424/756

(57) ABSTRACT

The present invention relates to combinations of extracts of various plants. A plant extract is obtained from unfermented rooibos by extraction with a mixture of water and alcohol. The rooibos extract contains a compound of formula I (aspacat). The compound of formula I is shown below:

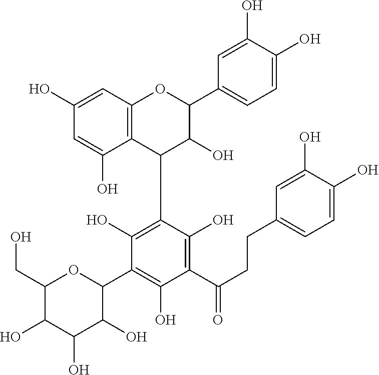

The extracts of other plants are obtained from fermented rooibos and/or green tea and/or curcuma and/or ginseng. The combinations can be used as drugs and/or food supplements for the prevention and/or treatment of dementia disorders.

The present invention relates to combinations of extracts of various plants which invariably comprise a rooibos extract together with at least one other extract of another plant. The extract combination can be used for preventing and/or treating dementia disorders.

In the widest sense, the combinations of extracts of various plants are used as foodstuffs, in particular as food supplement products and as drugs, particularly for treating neurological and psychiatric disorders of the central nervous system. The expression “pharmaceutically effective” also includes those effects which result in a subjective improvement in the mental state, in which case approval in terms of drug law does not have to be absolutely necessary.

Rooibos (Latin: Aspalathus linearis) grows only in South Africa and is presently the only plant known worldwide which contains the particularly strong antioxidant substance aspalathin, a flavonoid. Rooibos also contains further flavonoids, such as C-glycosyl flavones (inter alia orientin, isooreintin), flavonol-3-O-glycosides (inter alia quercetin, quercitrin, isoquercitrin, rutin) and glucosides, in particular C-glycosides and chalcones, such as nothofagin and aspalathin.

Compared to the fermented product, unfermented “green” rooibos is characterised by a higher content of polyphenols, in particular aspalathin, and by a higher antioxidant activity. The effect of the decrease in the antioxidant activity by the fermentation process can be observed equally for black tea and green tea (Bramati et al., J. Agric. Food Chem. 2003, 51: 7472-7474). Scientific experiments have shown that the antioxidant activity of rooibos tea is to be mainly attributed to the content of aspalathin. Investigations into the fermentation process of rooibos tea have shown that the content of aspalathin and nothofagin decreases during the fermentation process (Schulz et al., Eur. Food Res. Technol. 2003, 216: 539-543). Thus, it is possible to explain the lower antioxidant activity of fermented “red” rooibos tea compared to that of unfermented “green” rooibos tea.

Rooibos tea is widely used because of the aforementioned health-promoting flavonoids and due to its pleasant taste. Rooibos tea also contains phenolic acids, essential oil, vitamin C as well as numerous minerals, particularly iron.

A high content of aspalathin is required in order to achieve the highest possible antioxidant activity. In this respect, DE 10 2005 004 438 discloses a rooibos extract which, compared to the usual aspalathin content of 1 to 3% by weight, has an increased content of more than 5% by weight with at the same time a low chlorophyll content of less than 0.4% by weight. According to DE 10 2005 004 438, the rooibos extract is obtained by extraction from unfermented rooibos raw material using a mixture of 80 parts ethanol and 20 parts water. Due to its strong antioxidant, anti-irritant and antimicrobial action, it is stated that the rooibos extract with a high content of aspalathin is to be used in particular for cosmetic applications, for example as a care product for the hair, skin or mouth.

Quercetin is a further flavonoid contained in rooibos tea. This appears in a content of approximately 11 mg/100 g rooibos raw material and influences, for example the release of histamine in the human body, as a result of which allergic reactions can be alleviated. Quercetin is also capable of inhibiting the production of monoamine oxidase, which has an advantageous effect on mild depression and sleep disturbance (Plantextrakt, the nature network, 3^{rd} edition, dated Sep. 11, 2005; Plantextrakt GmbH).

A starting point of the present invention was the search for active substances for the treatment of disorders of the central nervous system, for example dementia, Morbus Parkinson, depression and painful conditions. These disorders are difficult to treat therapeutically and the drugs used for this purpose, such as tacrine, galantamine or nefopam have a broad spectrum of side effects.

In dementia disorders, a differentiation is made between Alzheimer's dementia, cerebrovascular dementia and dementias due to other causes (M. Prick, M. Parkinson, Chorea Huntington, and other, rarer causes), with Alzheimer's dementia being the most frequently occurring form. The former strict distinction between vascular dementia and Alzheimer's dementia has been abandoned in recent times.

According to the present state of knowledge, treatments for M. Alzheimer's dementia using the presently available monosubstances have not been particularly successful. Hitherto, cholinesterase inhibitors for mild to moderate development of Alzheimer's dementia states and the NMDA receptor antagonist memantine for moderate to severe development of Alzheimer's dementia states have been approved by the European and North American (USA and Canada) authorities; furthermore, in Germany an extract of ginkgo biloba is approved for Alzheimer's dementia. However, in clinical studies, although more or less significant results have been achieved using the mentioned substances, the extent of the effects has in no way been satisfactory, which is why the search for improvements in the effect continues and also why combinations with a respective second substance which have a different effect mechanism are being increasingly investigated.

Combinations are usually between cholinesterase inhibitors and a calcium/NMDA antagonist, but hitherto no significant therapeutic progress has been made here either in the sense of higher response rates and/or a stronger effect.

To date, no authority has granted authorisation for the initial stage of the disease since a positive use-risk ratio for any of the hitherto approved substances has not been demonstrated. There are a number of substances for which a significant prevention effect in respect of Alzheimer's dementia has been demonstrated by epidemiological investigations: statins, non-steroidal anti-inflammatories and oestrogens.

Etiopathologically, in M. Alzheimer's dementia, genetic causes are assumed in approximately 5 to 10% of all cases and a whole series of pathophysiological causes are discussed for the remaining 90 to 95% cases. The damage to the neurones which can be observed in any case can be rooted in many causes: (i) a disturbed cellular calcium homeostasis, (ii) an increase in the free oxygen radicals formed, (iii) acculumations of β-amyloid, (iv) lack of growth factors, (v) inflammatory processes, (iv) inductors of the programmed cell death (apoptosis), (vii) disturbance of the transmitter systems of the cholinergic, dopaminergic or glutamatergic signal paths, and (viii) secondary consequences of infections with Chlamydia pneumoniae and Herpes simplex or the cytomegalovirus. Hitherto, it has not been clarified which of these processes are connected together and how they are interconnected, and which are primary or secondary processes in respect of the course of the disease, although based on previous clinical studies it is clear that a medicinal influence on (vii) (cholinesterase inhibitors) and (i) calcium antagonists or NMDA antagonists does produce demonstratable therapeutic results which are, however, unsatisfactory. This is invariably against the background of significant side effects in the therapy.

Up until now, with the combination of chemical-synthetic drugs, the objective of a cure and also of palliation cannot be achieved in the long run because these substances each target a single pharmacophore, and even with the most up-to-date strategies for synthesising monosubstances with an incorporated combination effect, i.e. combining two pharmacophores in one molecule, it will be difficult to overcome the restriction to two pharmacophores and in addition a vast number of dosing, efficacy and compatibility questions will arise.

The development of synthetic active substance combinations will probably not happen due to the philosophy of drug manufacturers and of the authorising institutions and on account of the costs in demonstrating the efficacy, since drug legislation for chemical-synthetic active substances for combinations requires the advantages of the combination be demonstrated over the respective monosubstance; for three combination partners, this is extremely expensive. Consequently, we are unable to envisage a good or at least satisfactorily effective medicinal therapeutic approach.

It is a different matter in the field of plant active substances. Preparations of medicinal plants or useful plants are already substance mixtures which have a broader active substance spectrum, and the rational combination of a plurality of such preparations can cover a broad spectrum of effects. This maximises the likelihood of the patient responding to the medication and provides an effective and compatible possibility of treatment, which is also affordable.

WO 2007/057310 describes the use of rooibos extracts for protecting hair colour. This international patent application discloses, but not in detail, how the rooibos extracts are prepared. It is not specified whether the extracts are obtained using water and/or alcohols.

South African patent application 2003/3674 describes compositions which contain extracts obtained either from fermented and/or unfermented rooibos (Aspalathus linearis). The extracts described there are said to have an antioxidant action and they deactivate damaging free radicals.

It is therefore an object of the invention to provide combinations of extracts of various plants for the treatment and/or prevention of these disorders. In particular, these active substances or compositions should not have any or should only have negligible side effects so that they can also be used effectively as preventives.

This object is achieved by the subject matter of the claims.

The present invention relates to a combination of extracts of various plants which contains a combination of an extract obtained from unfermented rooibos together with at least one further extract, obtained from fermented rooibos, ginseng, green tea and/or curcuma. The combination according to the invention is preferably a combination of dry extracts. The extracts preferably have a moisture content of <5% by weight, more preferably of <4% by weight and most preferably of <2% by weight. The combination contains extracts of the following medicinal plants or food plants:
(1) green rooibos/unfermented rooibos-unfermented leaves and shoot tips of Aspalathus linearis (BURM. F.) R. DAHLGREN and
(2) a) green tea-unfermented tea leaves of Camellia sinensis (L.) KUNTZE; and/or
b) rooibos-fermented leaves and shoot tips of Aspalathus linearis (BURM. F.) R. DAHLGREN and/or
c) ginseng-root of Panax ginseng C. A. MEYER and/or
d) curcuma-root of Curcuma longa L. as a caffeine-free alternative to green tea.

A necessarily present component of the combination according to the invention of plant extracts is an extract of unfermented rooibos which contains at least 0.05% by weight, preferably at least 0.1% by weight, more preferably at least 0.18% by weight, particularly preferably 0.4% by weight and most particularly preferably at least 1% by weight of a compound of formula I

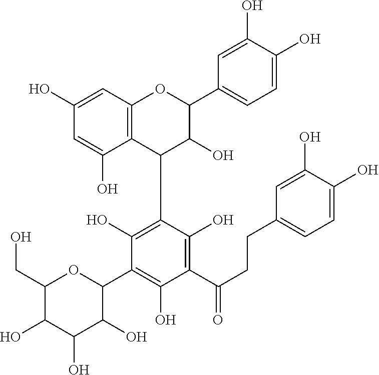

as well as the pharmaceutically acceptable salts, derivatives and esters thereof. In the following, the compound of formula I will also be abbreviated to aspacat. The preferred salts which are considered are the potassium, sodium, ammonium or gluconate salts. Esters of acetic acid, formic acid or propionic acid are preferred. Esters of fatty acids, such as C₁₀ to C₁₈ fatty acids are particularly preferred which can optionally also have one, two or three double bonds. Esters of fatty acids have hydrophobic radicals which influence the lipophilicity ratio of these esters. In this respect, preferred derivatives are coupling products of the compound of formula I to ferulic acid, quinic acid, caffeic acid, gluconic acid or chlorogenic acid. The compound of formula I is preferably used in its natural form or as a pharmaceutically acceptable salt, more preferably in its natural form according to formula I.

Preferred esters include formic acid ester, acetic acid ester, propionic acid ester, glutaric acid ester, tartaric acid ester or succinic acid ester. Preferred salts include the salts with cationic, organic or inorganic counterions, in particular alkali metal salts, alkaline earth metal salts, ammonium salts or also salts with pharmaceutically acceptable acids, such as succinates, citrates and tartrates.

A rooibos extract of unfermented rooibos is particularly preferably used which has a content of the compound of formula I of at least 1% by weight, preferably at least 1.5% by weight, particularly preferably at least 2.0% by weight, particularly preferably and most particularly preferably at least 5% by weight.

The rooibos extract which is used according to the invention is prepared in that
dried and crushed, unfermented rooibos raw material is extracted using an extracting agent consisting of alcohol and/or water for a predetermined extraction time at a temperature of up to 90° C., in particular up to 60° C.,
the extract is filtered and is subsequently concentrated to dryness under reduced pressure.

The extract used according to the invention of unfermented rooibos is preferably prepared such that leaves and shoot tips, which are dried particularly rapidly and carefully, of Aspalathus linearis (unfermented)=“green” rooibos are used as the primary drug with the lowest possible residual moisture content (<5%).

This drug is extracted using water or alcohol-water mixtures with at least 40% (vol./vol.) of water. Mixtures of 10-60%, preferably 10-50%, preferably 15-40%, more preferably 15-25% and particularly preferably 20% of methanol or 25-60%, preferably 30-50%, particularly preferably 30% (vol./vol.) of ethanol are preferably used. The remainder of the mixture is water.

Thus, an extract with a content of compound of formula I of from 0.05 to 0.4 can be obtained as a function of the charge used of the drug of up to 2.5-5% by weight. The total flavonoid content (total of aspalathin-type+rutoside-type+vitexin-type without compound according to formula I) is 17-30% with a ratio of vitexin-type (=C-glycosides) to rutoside-type: </=1.6, preferably ≦2.0 and a content of aspalathin-type: 14-25%.

The extract is preferably obtained using an optimised extracting agent, namely 20% methanol or 30% ethanol in water. Alternatively, the pure alcohol can initially be mixed with the drug and after a steeping phase of at least 30 minutes, the corresponding amount of water is added.

This extracting agent is optimised on the most complete extraction possible of the compound of formula I and at the same time on a high yield of total flavonoids.

The extract differs from hitherto commercially available extracts for internal use in tea drinks (purely aqueous extraction) in that it has higher contents of the compound of formula I and from the extract for external use in cosmetics (80% ethanol; Rapps) by the relationship of the flavonoid groups to one another. The 80% ethanol extract was optimised on the highest possible aspalathin content. The ratio of the three flavonoid groups (aspalathin-type, rutoside-type and vitexin-type=C-glycosides) is altered by the relatively lipophilic extraction compared to the starting state in the drug. This can be seen particularly clearly from the ratio of the vitexin-type flavonoids to the rutoside-type flavonoids.

In the case of the extract according to the invention, the objective is for the three flavonoid groups to be obtained as far as possible in the same ratio in the extract as they occur in the primary drug (while bearing in mind the natural variation limit of the drug, processing differences and differences from one charge to another). It is then possible to compare this ratio with the ratio in the tea drinks customarily prepared from high-quality green rooibos (of a low fermentation degree).

An extract which has an even higher content of the compound of formula I and total flavonoids can be obtained in that a high proportion of the substance is obtained from the previously described extract by a further purification using size-exclusion chromatography (Sephadex (or similar) column). By means of a chromatography step with Sephadex, it is possible to obtain an extract which has:

Compound of formula I content: >3%, preferably >5%.

Total flavonoid content (total of aspalathin-type+rutoside-type+vitexin-type without compound according to Formula I): >17%, preferably >35%

Ratio or vitexin-type (=C-glycosides) to rutoside-type: </=1.6, preferably </=2.0

Content of aspalathin-type: >20%, preferably >25%.

The extract of unfermented rooibos is rich in:

C-Glycosyl flavones, such as vitexin, iso-vitexin, orientin, isoorientin; Flavonol-3-O-glycosides, such as rutoside, quercetin, quercitrin, isoquercitrin and Chalcones, such as aspalathin, nothofagin.

The preferred extraction process can typically produce extracts which are summarised in Table 1.

The extracts obtained by the extraction process preferred according to the invention have in particular compounds which can be allocated to specific groups. These are:
a) substance of formula I (aspacat);
b) the group of chalcones, in particular quercetin, aspalathin and nothofagin, it being possible for aspalathin to have up to approximately 90% by weight of this group (denoted in Table I as substance group A);
c) the rutoside group includes flavonoids which contain quercetin as a component. Flavonoids which contain a sugar via a C—O—C linkage (quercetin is the aglycon of rutin); and
d) compounds of the so-called vitexin group. This is understood as meaning flavonoids which contain a sugar via a C—C linkage. Vitexin is the aglycon of apigenin, for example vitexin(apigenin-8-C-glucoside), isovitexin(apigenin-6-C-glucoside), orientin(luteolin-8-C-glucoside), isoorientin(luteolin-6-C-glucoside).

TABLE 1
Content of flavonoids-substance of formula I, rutoside group, substance A and vitexin
group based on the drug used and based on the differently prepared extracts
(starting from drug CH G310807SA).

| | Content [%] | | | | |
|---|---|---|---|---|---|
| | | | Substance A | Vitexin | Drug/Extract |
| | Substance I | Rutoside group | group | group | ratio |
| Drug | 1.09 | 0.56 | 5.81 | 0.87 | |
| (G310807SA) | | | | | |
| Extract (A): | 2.12 | 1.85 | 13.53 | 2.52 | 3.2:1 |
| Methanol 20% | (95% | (230% | 133% | (190% | |
| | 1.9 times) | 3.3 times) | 2.3 times) | 2.9 times) | |
| Extract (A): | 2.66 | 2.03 | 13.51 | 2.14 | 3.2:1 |
| Ethanol 30% | (144% | (262% | (132% | (145% | |
| | 2.4 times) | 3.6 times) | 2.3 times) | 2.5 times) | |
| Extract (B): | 2.57 | 2.45 | 15.65 | 3.14 | 3.4:1 |
| Methanol 20% | (136% | (338% | (169% | (261% | |
| | 2.4 times) | 4.4 times) | 2.7 times) | 3.6 times) | |
| Extract (B): | 3.02 | 2.26 | 14.51 | 2.85 | 3.2:1 |
| Ethanol 30% | (177% | (304% | (149.7% | (227% | |
| | 2.8 times) | 4.0 times) | 2.5 times) | 3.3 times) | |

The % contents are in each case averages of at least 2 extract preparations.
(A) The extraction was initially carried out for 10 minutes with the pure alcohol; water was subsequently added until the specified alcohol content was adjusted.
(B) The extraction was carried out from start to finish using an alcohol/water mixture with the specified alcohol content.
The values added in brackets in each case state the percentage by which or by how many times the corresponding flavonoid is contained in the respective extract compared to the drug.

The invention also relates to the use of the combination of plant extracts as a drug or food supplement. The food supplement or drug preferably contains the rooibos extract in a quantity of at least 25 mg, more preferably in a quantity of at least 50 mg, even more preferably in a quantity of at least 75 mg per dosage unit of the drug.

In a preferred embodiment of the invention, the combination with the rooibos extract having the increased proportion of compound according to formula I is used for the treatment of neurological and psychiatric disorders of the central nervous system, preferably for the treatment of dementias, Morbus Parkinson, depression and painful conditions, more preferably Alzheimer's disease.

The process for isolating the chemical compound is described in detail in Example 1, the structural formula being given in formula I. The compound of formula I has similarities both with the structure of aspalathin and with catechin(4α>2)-phloroglucinol (FIG. 1). Compared to the flavonoids known hitherto, the new compound according to formula I has a high molecular weight of 740.66 g/mol. Such high molecular natural substances are not usually used for active substance screening because, due to their molecular weight, they have difficulty passing through the blood-brain barrier. Thus, substances of this type tend not to be considered suitable for the brain site of action or for treating disorders of the central nervous system.

Surprisingly however, a “bias”-free investigation in tele-stereo EEG (electroencephalography) of rats exhibited a marked pharmacological activity on the central nervous system by the compound of formula I after oral administration. The pharmacological investigations surprisingly showed that the activity in the tele-stereo EEG model for rats produces dose-dependent changes in the EEG frequencies, as are known following the administration of conventional drugs for the treatment of dementias (for example galantamine or tacrine), Morbus Parkinson (L-DOPA) as well as painful conditions (for example nefopam).

In the context of the present invention, the pharmacological activity of the compound according to the invention of formula I was compared with the known constituents of the rooibos extract, such as aspalathin, catechin or (−)-epicatechin. The experiments unexpectedly show that the effect of the compound according to formula I cannot be achieved with approximately equimolar quantities of aspalathin, catechin or (−)-epicatechin, although the compound according to formula I has structural similarities with these natural substances. The new compound of the invention according to formula I has a higher pharmacological activity than these known constituents of the rooibos extract and is therefore particularly suitable for use as a medicament.

The isolation of this new compound with advantageous pharmacological activities makes it possible in particular to produce a medicament based on the compound of formula I combined with further active substances. These further active substances, namely the other plant extracts, have characteristics which advantageously influence the clinical picture in another way. Also responsible for this is the combination of this compound with other flavonoids and constituents contained in rooibos in the complete compound structure. Particularly suitable here are constituents which have an antioxidant action.

Constituents are particularly suitable here which have an antioxidant and/or neuroprotective and/or nerve function-activating (nerve stimulation transmission activating) and/or cognitive performance-improving and/or memory performance-improving effect.

The conventional agents against dementia such as tacrine and galactamine provide primarily for an improvement in nerve stimulation transmission, but they do not have a neuroprotective action, for example. The antioxidant action is just one aspect of the intended scope of action in this combination. What is remarkable about green rooibos is the cognitive improvement and memory improvement. Green rooibos acts synergistically with the other components. For example, green tea prevents plaque formation of proteins on the nerve cells and is also neuroprotective. Ginseng promotes a regeneration of nerve fibres. The combination of all these effects is a particular aspect of the invention. Furthermore, different substance groups were selected to provide the opportunity of achieving the best possible effects via different bioavailabilities in the tissues and via different effective mechanisms.

A discriminant analysis of the in vivo-data of the compound according to formula I showed, as mentioned above, a relationship with the drugs for the treatment of dementias, Morbus Parkinson, depression and painful conditions. Since these drugs have a broad spectrum of side effects, the use of a rooibos extract with the compound according to formula I is advantageous as a drug because natural substances can usually be expected to have a lower rate of side effects. Unexpectedly, the new substance evidently also crosses the blood-brain barrier. This is not generally commonplace for flavonoids.

Furthermore, a production process is provided for rooibos extracts and for the pharmacologically active compound identified here. The process according to the invention makes it possible to provide particularly suitable plant extracts for the treatment of the aforementioned disorders.

The rooibos extract prepared according to the invention has a content of the compound of formula I (\hat{=}aspacat) of at least 0.18% by weight, preferably at least 0.4% by weight, more preferably at least 1.5% by weight, even more preferably at least 2% by weight and most preferably at least 2.5% by weight.

In a particularly preferred embodiment, a rooibos extract is prepared according to the invention which contains at least 10, particularly preferably at least 20% by weight of the compound according to formula I.

In a further particular embodiment of the combination according to the invention, the total flavonoid content in the unfermented rooibos extract is at least 17% by weight.

A process according to the invention for the preparation of the compound according to formula I has the following steps (see also Example 1):
preparation of dried and crushed, unfermented rooibos raw material,
extraction of the prepared raw material using an extracting agent consisting of a mixture of an alcohol, preferably methanol and/or ethanol and water for a predetermined extraction time at a temperature of up to 90° C., preferably up to 60° C.,
filtration of the extract,
concentration of the filtered extract under reduced pressure.

Further purification of the extract in up to three steps can then be carried out, said steps being:
coarse purification by chromatography on a Sephadex LH20 column
fine purification by chromatography on a further Sephadex LH20 column
separation on silica gel, preferably of a lipophilic c18-HPLC column.

If preparations with an increased content of compounds of formula I are desired, it is also possible to separate the extract using only one chromatography column.

The moisture content of the prepared rooibos raw material is preferably from 4 to 5% or less, as this prevents autofermentation of the starting material. The rooibos raw material is preferably immediately subjected to extraction and is not stored for a prolonged period of time.

According to a preferred embodiment of the process according to the invention, the extracting agent used is an alcohol/water mixture in a ratio of 20:80 to 50:50. Alcohols such as methanol, ethanol, propanol or propan-2-ol are preferably used. A 50:50 methanol/water mixture is preferably used if a particularly high proportion of the compound of formula I is desired. In this preferred embodiment of the process according to the invention, the ratio of raw material to extracting agent is preferably approximately 1:6, and the extraction step is preferably carried out at elevated temperature (above 40° C.) for 1 hour, but is also possible at room temperature and for a period of, for example 2 to 5 hours. When there is a low alcohol content of 20 to 30% (vol./vol.), a higher content of flavonoids is obtained.

The filtered extract is preferably concentrated under a pressure of less than 300 mbar. During the concentration step, the temperature is preferably at most 40° C.

A rooibos extract according to the invention is prepared according to the process described above, the rooibos extract being obtained after concentration to dryness (without subsequent chromatographic purification).

The measurement method for determining the content of rooibos extract in the compound according to formula I is described in detail in Example 4.

The compound according to formula I, the pharmaceutically acceptable salts, derivatives and esters thereof and the rooibos extract according to the invention are particularly suitable for the prevention and/or treatment of disorders of the central nervous system, preferably dementias, Morbus Parkinson, depression, painful conditions and as cell protecting antioxidants or “free-radical scavengers”. The treatment of Morbus Alzheimer's is particularly preferred. The extract according to the invention is used to improve the mental/memory performance even if a disorder/dementia is (still not) identifiable.

According to the invention, the rooibos extracts with the compound of formula I are used directly as such or also as the esters or derivatives thereof combined with further active substances. Suitable derivatives, salts, complexes and esters as well as the preparation thereof are known to a person skilled in the art. The preparation of pharmaceutically acceptable salts is also known to a person skilled in the art. All conventional pharmaceutically acceptable acids and anions are included as salt formers. Furthermore, couplings to acids such as ferulic acid, quinic acid, caffeic acid, gluconic acid, chlorogenic acid and related compounds are possible. A coupling to gluconic acid is particularly preferred. The coupling products of the compound according to formula I to the above acids are denoted as pharmaceutically acceptable derivatives. However, the compound is preferably used as a molecule according to formula I.

The rooibos extract according to the invention can be processed in a manner known per se into drugs and/or food supplements which have health-promoting characteristics. The rooibos extract according to the invention combined with other plant extracts can be formulated as, for example, tablets, capsules, pills, coated tablets, granules, powders, lozenges and liquid forms of administration such as drinks or soluble tea extract. It is preferably also used in food supplements.

The food supplements or drugs are preferably administered orally, but it is also possible to administer them topically, parenterally, intravenously, intramuscularly, subcutaneously, nasally, inhalatively, rectally or transdermally, for example.

The rooibos extract according to the invention and the drugs and food supplements according to the invention can also preferably contain further active substances which enhance the action of the rooibos extract or of the compound of the invention according to formula I or the salts thereof or additionally have a positive effect on the symptoms or conditions which occur in the mentioned disorders (for example: further free-radical scavengers, various enzyme-inhibiting substances, vitamins, lecithins, omega-3-fatty acids and substances which have a positive effect on brain function), and also ascorbic acid.

In addition to the rooibos extract of unfermented drug, the combination according to the invention also contains at least one, preferably at least two and particularly preferably at least three extracts of other plants, the respective quantity ranging in particular within the following limits, based on one dosage unit:
Green rooibos extract: 10-2000 mg, preferably 100-600 mg, more preferably 200-400 mg, particularly preferably 50-150 mg, most particularly preferably approximately 75 mg
Green tea extract: 50-2000 mg, preferably 50-500, preferably 100-200, more preferably 75-200 mg, particularly preferably approximately 100 mg
Fermented rooibos extract: 10-2000 mg, preferably 100-600 mg, more preferably 200-400 mg, particularly preferably 50-100 mg, most particularly preferably approximately 75 mg
Ginseng extract: 10-1000 mg, preferably 50-300 mg and particularly preferably 50-100 mg
Vitamin C as stabilising additive and as a means for improving the bioavailability: 20-1000 mg, preferably 20-500 mg, more preferably 20-100 mg.

The combination according to the invention always has a dry extract of unfermented (“green”) rooibos (Aspalathus linearis) containing aspacat, the compound of formula I, as described above. Like the primary drug, this extract contains the following main flavonoids:

aspalathin, nothofagin, rutoside, quercitrin, isoquercitrin, quercetin, vitexin, orientin, isoorientin.

The content and the ratio of the other flavonoids can vary as a function of the degree of concentration. Although extracts which are strongly concentrated with aspacat contain further flavonoids of rooibos, this content is significantly reduced and is no longer in the original quantity ratios.

In the extract, the ratio of the flavonoids to one another is preferably approximately the same as that in the drug. This can be measured in the ratio/factor which is obtained when the content of all flavonoids with a UV spectrum similar to orientin/vitexin (C-glycosides) is divided by the content of all flavonoids with a rutoside similar UV spectrum (quercetin derivatives). Criterion: this factor is for the most part </=1.6 or </=2.0.

The following plant extracts can be combined with the dry extract of green rooibos:

1) dry extract of rooibos (fermented, Aspalathus linearis)

Extracting agents are water and mixtures of water with alcohol, it being possible to use as alcohol: ethanol, methanol, propanol-1, propanol-2. Ethanol or methanol is preferably used in a quantity of 0-30%, remainder: water. In the constituents, aspalathin should be present in a quantity of >0.4%, preferably >3.5%.

Like the primary drug, the extract contains the following flavonoids:

aspalathin, rutoside, quercitrin, isoquercitrin, quercetin, vitexin, orientin, isoorientin, eriodyctyol-glycosides.

2) dry extract of green tea (Camellia sinensis)

Extracting agent: water and mixtures of water with alcohol. After-treatment of the primary extract (solid or liquid) with a ketone (ethyl acetate, acetone, methyethyl ketone) is possible. The following can be used as alcohol: ethanol, methanol, propanol-1, propanol-2, preferably ethanol or methanol in a quantity of 0-90%, preferably 40-85%, the remainder being water.

80% ethanol is particularly preferred. In this respect, extracts containing caffeine are obtained. If decaffeinated green tea leaves are used as the starting material, caffeine-free extracts are obtained.

Particular constituents are:

Catechins: epigallocatechingallate (EGCG), epicatechingallate, catechin, gallocatechin, epigallocatechin, inter alia.

Theanine, caffeine, theophylline, theobromine.

Particular constituents:

EGCG: >10%, preferably: >25% (calculated as EGCG)

Total catechins (calculated as catechin/epicatechin): >15%, preferably: >40%

Theanine: >0.1%, preferably: >1%

Extracts containing caffeine:

Caffeine: >2%, preferably: >5%

3) Extract of curcuma (Curcuma longa and/or Curcuma xanthorrhiza or mixtures of the two)

Extracting agent: mixtures of water with alcohol and/or acetone, pure alcohols or one of the following ketones: ethyl acetate, acetone, methylethyl ketone, and supercritical carbon dioxide.

The after-treatment of the primary extract (solid or liquid) with a ketone (ethyl acetate, acetone, methyethyl ketone) is possible. The following can be used as alcohol: ethanol, methanol, propanol-1, propanol-2.

Ethanol 96-200%, acetone are preferred.

Particular constituents:

Curcuminoids: curcumin, desmethoxy-curcumin, bisdesmethoxy-curcumin and optionally essential oil.

Contents in the extract: total curcuminoids: >1%, preferably >10% particularly preferably: >25% curcuminoids.

4) Dry extract of ginseng (Panax ginseng). A preferred extracting agent is an ethanol-water mixture with 40% ethanol.

Extracting agent: water and mixtures of water with alcohol.

In principle, the following alcohols can be used: ethanol, methanol, propanol-1, propanol-2.

Ethanol or methanol 5-96% are preferred.

Particularly preferred: ethanol 40-80%.

Particular constituents:

Triterpensaponins (ginsenosides) 2 groups:

Protopanaxadiols: ginsenoside Rb1, Rb2, Rc, Rd inter alia.

Protopanaxatriols: ginsenoside Rg1, Rg2, Rf inter alia.

Content in the extract:

Total ginsenosides (HPLC calculated as Rb1): >1%, preferably >2%, particularly preferably: >5%

Furthermore, pharmaceutically acceptable auxiliaries and carriers can be used. Suitable auxiliaries are known to a person skilled in the art and include, for example fillers, disintegrating agents, lubricants, binders, wetting agents, etc.

Suitable lubricants include, for example silicate, talc, stearic acid, magnesium or calcium stearate and/or polyethylene glycols.

Binders which can be used are, for example starches, gum arabicum, gelatine, methyl cellulose, carboxymethyl cellulose or polyvinyl pyrrolidone.

Decomposing agents are, for example starch, alginic acid, alginates or sodium starch glycolates, foaming mixtures.

Wetting agents which can be used are, for example lecithin, polysorbate or lauryl sulphates.

Furthermore, dyes and sweeteners can also be contained in the formulations.

The pharmaceutical preparations can be prepared in a known manner, for example by mixing, granulating, tabletting or by sugar-coating or cover coating processes.

The liquid dispersions and/or solutions for oral administration can be drinks, drops, syrups, emulsions and suspensions, for example.

As carrier, the syrup can contain saccharose or saccharose with glycerine and/or mannitol and/or sorbitol, for example.

As carrier, the suspensions and emulsions can contain a natural resin, agar, sodium alginate, pectin, methylcellulose, carboxymethyl cellulose or polyvinyl alcohol, for example.

The suspensions or solutions for intramuscular injection can contain, together with the active substance, a pharmaceutically acceptable carrier, for example sterile water, olive oil, ethyl oleate, glycols, for example propylene glycol and, if required, a suitable quantity of lidocaine hydrochloride.

The solutions for intravenous injection or infusion can contain sterile water for example as carrier or they can preferably be present in the form of sterile, aqueous, isotonic salt solutions.

The suppositories can contain, together with the active substance, a pharmaceutically acceptable carrier, for example cocoa butter, polyethylene glycol, a polyoxyethylene sorbitol fatty acid ester or lecithin.

Compositions for topical application, for example creams, lotions or pastes can be prepared by mixing the active substance with a conventional oil-containing or emulsifying carrier.

The combination according to the invention can contain an unfermented rooibos extract and the compound of the invention according to formula I or the salts, derivatives and esters thereof in usual quantities in the food supplements or drugs. Preferably 0.001 to 10% by weight of the compound of the invention according to formula I or the salts thereof, more preferably 0.1 to 7% by weight and particularly preferably 1 to 5% by weight are used in solution. In a particular embodiment, 0.02 to 1% by weight of the compound of the invention according to formula I or the salts thereof are used in solution.

According to the invention, the unfermented rooibos extract is preferably used in quantities which correspond to a quantity of the compound of formula I of 1 to 1000 mg, more preferably 10 to 600 mg, even more preferably 50 to 400 mg and most preferably 50 to 250 mg.

When a rooibos extract is used which has a very high content of the compound according to formula I, such an extract can be used in drugs and food supplements in a quantity of between 3 and 600 mg, preferably between 5 and 100 mg and particularly preferably between 10 and 50 mg per daily dose.

The food supplements (foodstuffs) and drugs described above can be prepared by conventional methods and administered in a pharmaceutically suitable form.

The solid food supplements and drugs preferred according to the invention can also preferably contain 1 to 50% by weight, more preferably 1 to 20% by weight, particularly preferably 1 to 10% by weight of fillers.

As fillers, one or more compounds can be used which provide part of the material for attaining the necessary and desired tablet or capsule mass. Substances which can be used include, inter alia, microcrystalline cellulose in different particle sizes, in particular with an average particle size within a range of 20 μm to 200 μm, in particular within a range of 50 μm to 150 μm, for example approximately 100 μm, such as the known Avicel products, for example Avicel PH-101 and PH-102. Further suitable fillers include, for example corn starch, potato starch, lactose, cellactose (a mixture of cellulose and lactose), calcium phosphate, dextrose, mannitol, maltodextrin, isomalt, optionally also sorbitol and saccharose. If direct compaction is intended, when selecting the fillers it should be ensured that qualities are used which are suitable for direct compaction of tablets. In the case of commercial products, this is specified by the manufacturer in each case or can be checked by means of simple tests. The most preferred filler is microcrystalline cellulose (commercial products are Avicel, Vivapur and Emcocel, for example).

Suitable disintegrating agents are known in the prior art. Disintegrating agents are frequently also called by the English name “disintegrants”. Disintegrants preferred according to the invention are, for example Crospovidone (Kollidon CL) and starch or pre-gelatinised starch, in particular the commercial product “Starch 1500”. Further suitable starches can be obtained commercially, for example under the names Lycatab PGS, Prejel and Sepistab ST 200. The known so-called “super disintegrants” can also be used, such as croscarmellose sodium (for example Ac-Di-Sol, inter alia) and carboxymethyl starch (for example Explotab, primojel, inter alia). Starches such as starch 1500 are particularly preferred.

The content of disintegrant is usually from 1 to 25% by weight, preferably 1 to 20% by weight, in particular 2 to 15% by weight. Suitable ranges for the content of disintegrant are also, for example 2 to 5% by weight or 15 to 20% by weight, depending on the disintegrants, fillers and other additives used.

According to the invention, as lubricant, the composition can contain one or more compounds which promote the preparation and processing of the tablets. Lubricants which can be used are, inter alia, stearic acid and derivatives thereof such as calcium stearate, and in particular sodium stearyl fumarate (which for example is commercially available under the name Pruv) and magnesium stearate, glycerolmono-, di- and in particular tristearate, hydrogenated vegetable oil (for example Lubritab, Dynasan, Sterotext) or a polyethylene glycol (for example Lutrol, Carbowax).

The content of lubricant is usually from 0.1 to 4% by weight, preferably 0.2 to 4% by weight.

The pharmaceutical composition according to the invention can optionally contain one or more flow regulators. Suitable flow regulators include magnesium trisilicate, talc and in particular silicon dioxide (for example Aerosil). If the composition includes a flow regulator, this is usually present in a quantity of from 0.5 to 5% by weight, preferably 1 to 4% by weight, in particular 2 to 3% by weight.

The pharmaceutical compositions according to the invention can also contain stabilisers for the active substance, such as ascorbic acid, citric acid, tartaric acid, lactic acid etc., preferably ascorbic acid and/or citric acid. The content of stabiliser (if present) is usually within a range of from 0.1 to 10% by weight, preferably 0.5 to 10% by weight, preferably 1 to 3% by weight.

The pharmaceutical compositions according to the invention can contain further conventional pharmaceutically acceptable additives and auxiliaries, but they preferably do not contain any further auxiliaries in addition to those mentioned above (filler, disintegrant, lubricant and optionally flow regulator and stabiliser).

Some fillers, such as microcrystalline cellulose can also be used as binders. Therefore, fillers with a binder function are also included among the fillers in the context of the present invention.

If the pharmaceutical composition according to the invention is present in tablet form, it can be film-coated with one or more coating agents. Coating agents which can be used are shellac or shellac mixtures, hypromellose (hydroxypropylmethylcellulose), polyvinyl alcohol, sodium carboxymethylcellulose and various methacrylic acid polymers (Eudragit), with hypromellose and in particular Eudragit, shellac or shellac mixtures being preferred. The tablets are coated in the conventional manner. The coating can contain, apart from the coating agent, further conventional components of tablet coatings such as plasticisers, pigments, pore-formers or suspension stabilisers, for example polyethylene glycol (PEG), talc or titanium dioxide and optionally also lactose.

The tablet weight is not restricted in particular; tablets weighing 100 mg to 500 mg are usual when pure active substances are used and from 500 mg to 1500 mg when extracts and plant powders are used. Quantities of 100 mg to 1000 mg are used in capsules.

The dosage unit of the drug or food supplement can contain for example:
for peroral drug forms:
   preferably 1 to 1000 mg, more preferably 40 to 800 mg, particularly preferably 150 to 500 mg, even more preferably 300 to 600 mg of rooibos extract per daily dose. When the compound according to formula I is used as well as the pharmaceutically acceptable salts, derivatives and esters thereof, 1/100 to 1/20 of the above quantities are used.
   The daily dose can be given, for example in 1 to 3 single doses, preferably in two single doses. It can also be provided that 1-10 single doses of rooibos extract containing the compound according to formula I are administered daily.
for parenteral drug forms (for example intravenous, subcutaneous, intramuscular):
   preferably 3 to 60 mg, particularly preferably 10 to 30 mg of active substance per daily dose.
   The daily dose can be administered for example in 1 to 3 single doses, preferably in one single dose.
for drug forms to be applied rectally:
   preferably 40 to 80 mg, particularly preferably 60 mg of active substance according to formula I per daily dose.
   The daily dose can be administered for example in 1 to 3 single doses, preferably in one single dose.
For drug forms to be applied to the skin and mucous membranes (for example solutions, lotions, emulsions, ointments etc.):
   preferably 40 to 80 mg of active substance, particularly preferably 60 mg of active substance per single dose. If the content of compound according to formula I is based on the finished solution, lotion, emulsion or ointment, the percentage by weight based on such ointment-type drugs is between 0.05 and 20% by weight, preferably between 0.2 and 1% by weight of compound of formula I based on the cream-type preparation.
   The daily dose can be administered in 1 to 6 in single doses, preferably in 1 to 3 single doses.

10-2000 mg, preferably 10-1000 mg, preferably 10-500 mg of combination according to the invention can be used per dosage unit.

### DESCRIPTION OF THE FIGURES

FIG. 1 shows the structural formula of aspalathin (1) and catechin(4α>2)-phloroglucinol (2).

FIG. 2 shows the numbering of the atoms in the compound according to formula I.

FIG. 3 shows a UV spectrum of the compound with formula I.

FIG. 4 shows a UV spectrum of aspalathin.

FIG. 5 shows a UV spectrum of rutoside.

FIG. 6 shows a UV spectrum of orientin.

FIG. 7 shows a UV spectrum of homoorientin.

FIG. 8 shows a UV spectrum of vitexin.

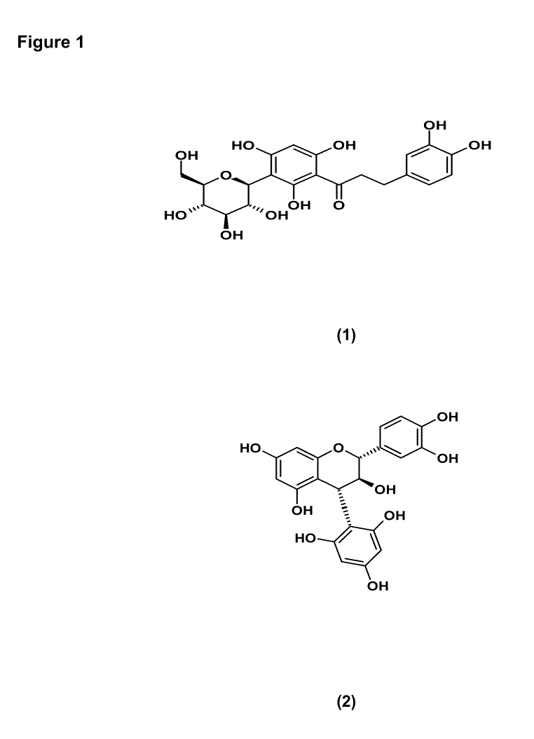

### EXAMPLE 1

### Isolation of the Compound According to Formula I

For preparation from the drug, unfermented green rooibos, manufactured by Rooibos Ltd. Clanwilliam South Africa is used.

As starting material, unfermented and crushed leaves and/or shoot tips of Aspalathus linearis which are carefully dried to a moisture content of less than 10% (preferably less than 4%) are used.

This raw material is extracted with a mixture of methanol and water in a 50:50 ratio (parts by volume) at 60° C. for 1 hour with rotation, the ratio of raw material to solvent being 1:7. Thereafter, the liquid is filtered off from the plant parts and the plant parts are extracted again in the same manner and filtered.

The two filtrates are combined and freed from methanol under reduced pressure (220 mbar) and at 55° C. The remaining aqueous solution is diluted with water to five times the weight of the quantity used of dried plant parts and is subjected to a liquid-liquid partition.

3 litres of the aqueous solution are shaken out four times with in each case 1.5 L of water-saturated n-butanol and the combined butanol phases are brought to dryness under reduced pressure. The yield amounts to approximately 10% of the quantity used of dried plant parts.

There then follows an initial coarse separation and thereafter a fine separation of the butanol extract.

Coarse separation:

Approximately 50 g of butanol extract are chromatographed on a Sephadex LH20 column (6 cm internal diameter and 80 cm filling height=2260 ml Sephadex LH20) with 50% by volume of methanol. For this, the 50 g of butanol extract are dissolved in 400 ml of mobile phase and introduced onto the separation column. The column is washed with a flow of 1.8 ml/min until 3 L of eluate have trickled off. The column filling is removed after the remaining mobile phase has completely trickled off and is stirred (extracted) for 10 minutes in 3 L of 100% methanol. The stationary phase is filtered off and the eluate is dried. The residue amounts to approximately 0.5 to 1% of the quantity used of plant parts =methanol extract.

First fine separation:

Approximately 4 g of methanol extract are chromatographed on a Sephadex LH20 column (3.5 cm internal diameter and 50 cm filling height=480 ml Sephadex LH20) with 80% by volume of methanol. For this, the 4 g of butanol extract are dissolved in 40 ml of mobile phase and introduced onto the separation column. The column is operated with a flow of 2.4 ml/min and fractions each of 10 min duration=24 ml eluate are collected. The desired substance is found in fraction numbers 48-65. The yield with 4 g of methanol extract is approximately 0.5 to 1 g.

Second fine separation:

Separating column: 250×30 mm

Stationary phase: Reprocil C18 Aqua 10 μm

Mobile phase: methanol 35% (v/v)

Flow: 1.5 ml.min

Fraction size: 10 min.=15 ml

Substance I is found in fractions 41 to 52

The combined fractions 41 to 52 are lyophilised.

Yield approximately 125 mg of substance I from 4 g of methanol extract Chromatographic purity approximately 97% (HPLC).

### EXAMPLE 2

### Structural Elucidation of the Compound According to Formula I

The compound obtained by column chromatographic separation was characterised by different methods.

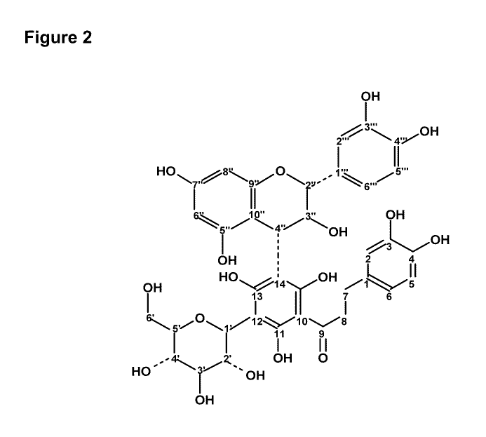

FIG. 2 shows the numbering of the atoms in the compound of the invention according to formula I.

### EXAMPLE 3

### Measurement Method for Determining the Content of the Compound According to Formula I

The content of the compound according to formula I in rooibos and preparations of rooibos (tea, extract, tablets) is determined by HPLC/DAD according to the external standard method. To determine the content, the substance of the compound according to formula I is used as external standard. Evaluation is carried out at a detection wavelength of 280 nm. To prevent oxidation processes in the analysis solution, ascorbic acid is added to the samples.

The HPLC device preferably used is Acquity UPLC/Alliance 2695; Detector: DAD, 200 to 400 nm; Column: Reprosil-Pur ODS-3, 125×3 mm, 3 μm, manufactured by Dr. Maisch; Column temperature: 60° C.

Eluent:Eluent A: water/formic acid 100/0.2 (v/v);
Eluent B: acetonitrile/methanol/water/formic acid 50/25/25/0.2 (v/v/v/v).

Injection volume: 20 μL.

Running time: 95 min.

Retention time of the compound according to formula I: 39.5 min.

TABLE 2
(Gradient):

| | | | | Gradient |
|---|---|---|---|---|
| Time (min.) | Flow (ml/min.) | Eluent A % | Eluent B % | increase |
| 0 | 0.3 | 100 | 0 | |
| 40 | 0.3 | 70 | 30 | linear |
| 60 | 0.5 | 20 | 80 | linear |
| 80 | 0.5 | 20 | 80 | linear |
| 81 | 0.3 | 100 | 0 | |
| 95 | 0.3 | 100 | 0 | |

The standard solution used:

1 mg of the compound according to formula I, precisely weighed, and approximately 20 mg of ascorbic acid are dissolved with 2 ml of methanol and made up to 20.00 ml with water.

Desired concentration: 0.05 mg/mL of the compound according to formula I and 1 mg/mL of ascorbic acid.

The analysis solution used:

Drug Formulation:

The sample to be investigated, for example a tablet is pulverised in a powder mill and sieved using a sieve with a mesh width of 250 μm.

Approximately 0.5 g of pulverised sample together with approximately 50 mg of ascorbic acid are weighed precisely into a 50 mL measuring flask, mixed with 10 mL of methanol at 40° C. and extracted for 10 minutes in an ultrasonic bath at 40° C. The flask is then filled up to the mark with water, is shaken vigorously and the mixture is extracted again for 10 min in the ultrasonic bath at 40° C. After cooling, the flask is optionally filled with water up to the mark and the solution is centrifuged for 5 min at 9300 g. The supernatant is filtered directly via a 0.45 μm membrane filter into a small amber glass bottle for the automatic sample injector of the HPLC installation

Dry Extract Extracting Agent Water:

Approximately 125 mg of rooibos extract, together with approximately 25 mg of ascorbic acid are weighed into a 25 mL measuring flask, mixed with approximately 22 mL of water, shaken vigorously, optionally treated in the ultrasonic bath and filled to the mark with water.

Dry Extract Extracting Agent Not Water:

Approximately 125 mg of rooibos extract, together with approximately 25 mg of ascorbic acid are weighed into a 25 mL measuring flask, mixed with approximately 2.5 mL of methanol and treated for 10 min in the ultrasonic bath. The flask is then filled with water to the mark, vigorously shaken and the mixture is treated again in the ultrasonic bath for 10 min.

Evaluation:

Standard solution and analysis solution are chromatographed directly after one another under the same conditions. The UV spectra of the reference substance are compared with the substance which was detected in the analysis chromatogram for the same retention time and are calculated according to the following calculation formula with conformity of the peaks as compound according to formula I:

$$Content [ \% ] = \frac{\begin{matrix}Analysis peak area \cdot Analysis solution dilution ( mL ) \cdot \\ Standard weighed quantity ( mg ) \cdot 100\end{matrix}}{\begin{matrix}Standard peak area \cdot Standard solution dilution ( mL ) \cdot \\ Analysis weighed quantity ( mg )\end{matrix}}$$

### EXAMPLE 4

### Rooibos Extract with a High Total Flavonoid Content

a) Preparation Process

10 g of dried and pulverised, unfermented rooibos raw material are mixed with 0.2 g of ascorbic acid and 60 ml of ethanol absolute and macerated for 10 min at 40° C. using ultrasound. Thereafter, 140 ml of demineralised water are added, shaken vigorously or stirred and then macerated again for 10 min at 40° C. using ultrasound. The entire mixture is then centrifuged (approx. 9000×g), the supernatant is filtered off, the residue is mixed with 100 ml of 30% (v/v) and then macerated again for 10 min at 40° C. using ultrasound. Centrifugation is then carried out and the supernatant is filtered off. The combined filtrates are concentrated under reduced pressure (max. 300 mbar) to approximately 10-20 ml and then freeze-dried. Spray-drying is a suitable alternative to freeze-drying. In the event of spray-drying, the concentration procedure is continued until a viscosity of approximately 130 mPascal is attained. This solution is then spray-dried. If necessary, the usual auxiliaries (Aerosil, lactose, maltodextrins) can be added to the solution before spray-drying.

This process produces an “Extract (A): ethanol 30%” according to Table 1.

b) Analysis

0.5 g of extract (precisely weighed), together with approximately 50 mg of ascorbic acid is weighed into a 50.0 mL measuring flask, mixed with 10 mL of methanol (40° C.) and extracted for 10 min in an ultrasonic bath at 40° C. The flask is then filled with water up to the mark, shaken vigorously and the mixture is extracted again for 10 min in the ultrasonic bath at 40° C. After cooling, the flask is optionally filled with water to the mark and the solution is centrifuged for 5 min at 9300×g.

1 ml is removed from the supernatant, filtered using a spray filter into a vial (amber glass) and measured by HPLC.

In the case of the drugs, the remaining solution (49 ml) was rotated off by a rotavapor and after freeze-drying, the residue was determined.

4a) Substance 1

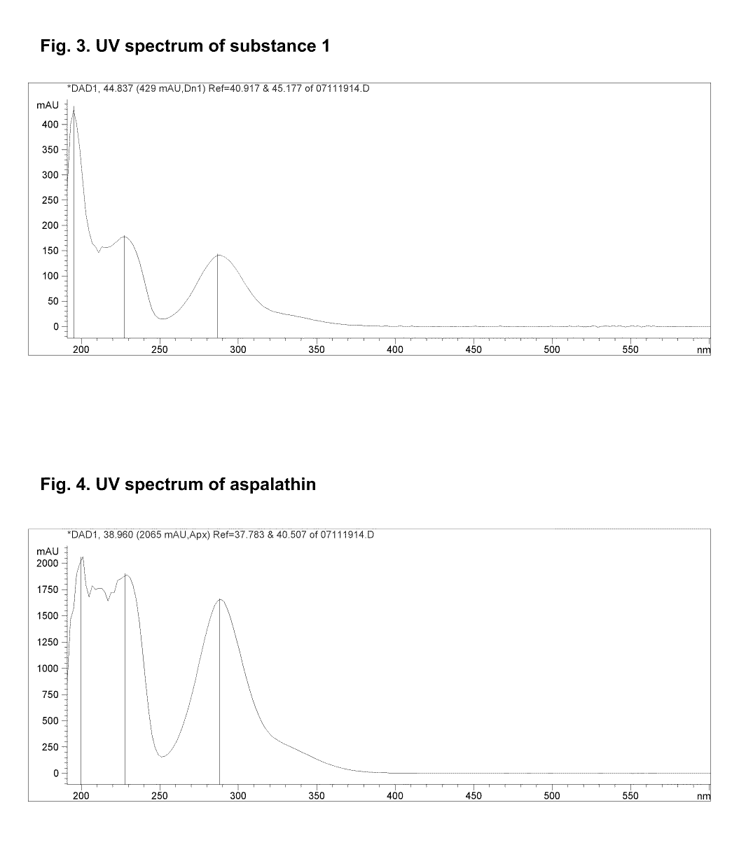

The substance with formula I in the chromatogram of extract 1 (G110907SA) was identified by an available comparison spectrum and by the UV spectrum (FIG. 3).

The content of substance 1 in different extracts was calculated using the known content in extract 1 (G110907SA) (0.95%) by means of the following formula (F-area, V-volume, m-mass, g-content, Ana-analysis, St-standard):

$${g}_{substance 1} ( \% ) = \frac{{F}_{Ana} {V}_{Ana}}{{m}_{Ana} {relF}_{St}} 100 \%$$

The relative area relF was calculated as follows:

$$relF = \frac{100 {F}_{St}}{{V}_{Extract} {m}_{Extract} 0.95} = 12.52 \pm 0.04$$

4b) Flavonoids of Substance Group A

Flavonoids of substance group A are characterised by UV maxima at 287 nm and 228 nm. All peaks which exhibit a substantial conformity with this spectrum (FIG. 4) are included in this group and the content is calculated using the following formula:

$${g}_{group A flavonoids} ( \% ) = \frac{{F}_{Ana - group A} {V}_{Ana} {m}_{rutoside} {k}_{f}}{{m}_{Ana} {F}_{rutoside} {V}_{rutoside}} 100 \%$$

Rutoside was used as standard, the correction factor k_{f} is 0.4.

4c) Flavonoids of the Rutoside Group

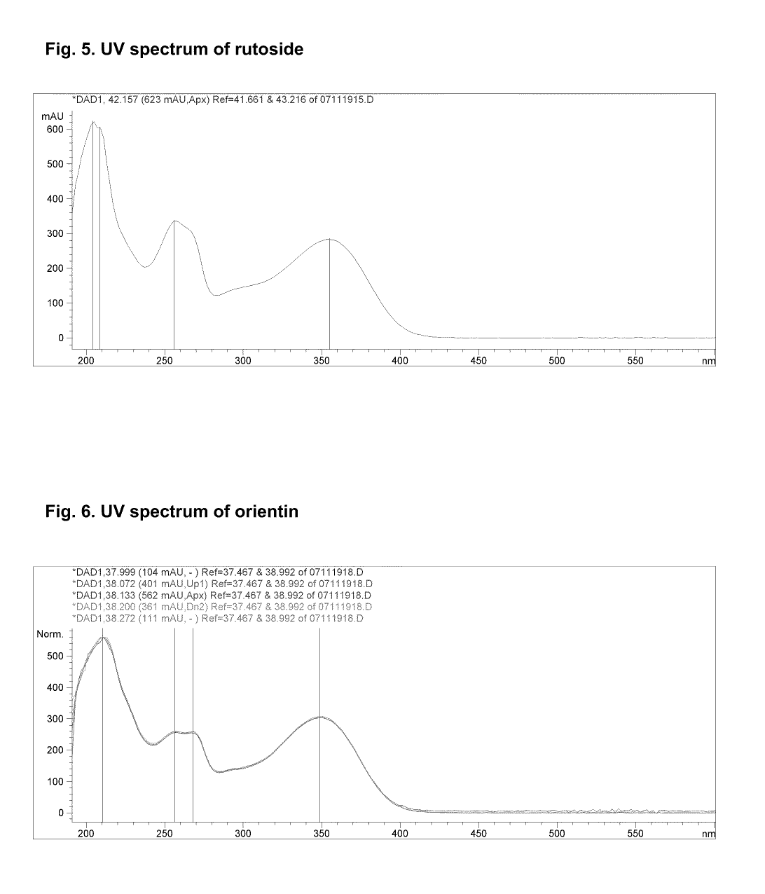

Flavonoids are allocated to this group using a rutoside comparison spectrum (FIG. 5). The content is calculated by means of the following formula:

$${g}_{rutoside flavonoids} ( \% ) = \frac{{F}_{Ana - rutoside} {V}_{Ana} {m}_{rutoside}}{{m}_{Ana} {F}_{Rutoside} {V}_{Rutoside}} 100 \%$$

4d) Flavonoids of the Vitexin Group

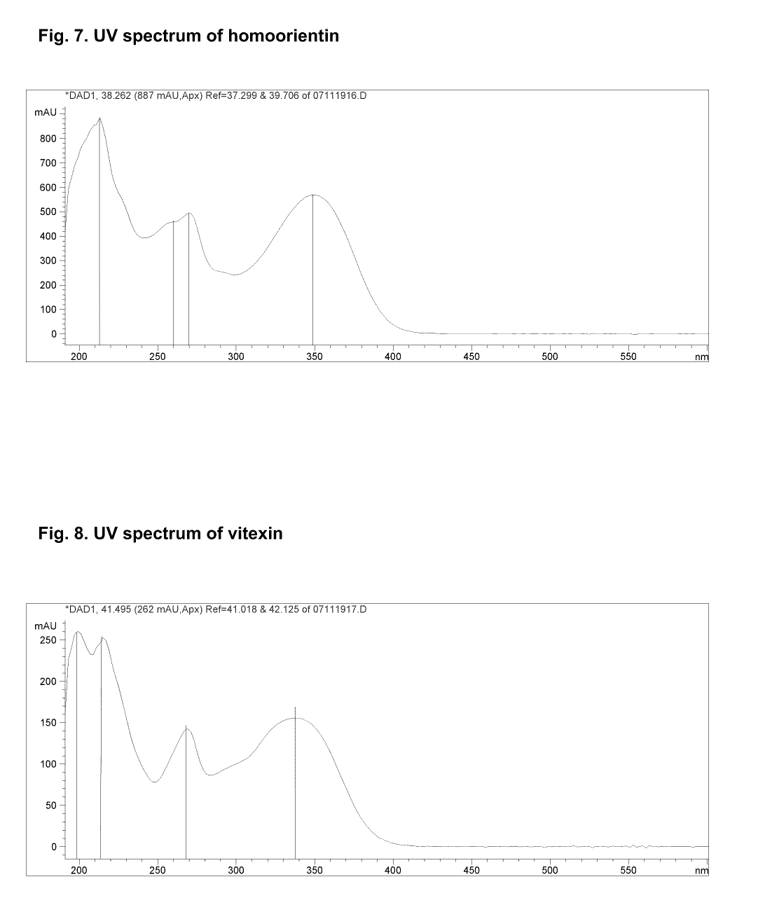

Flavonoids are allocated to this group using a vitexin comparison spectrum, with orientin and homoorientin also belonging to this group (FIG. 6 to FIG. 8). As standard, homoorientin is used instead of vitexin, since vitexin (as well as orientin) exhibited solubility problems during the preparation of the standard solutions. The content is calculated using the following formula:

$${g}_{vitexin flavonoids} ( \% ) = \frac{{F}_{Ana - vitexin} {V}_{Ana} {m}_{homoorientin}}{{n}_{Ana} {F}_{homoorientin} {V}_{homoorientin}} 100 \%$$

### EXAMPLE 5

### Preparation of a Concentrated Rooibos Extract with an Increased Content of Compound I

Implementation of the Sephadex Column

750 mg of an extract according to Example 4 were dissolved in 6 ml of MeOH, centrifuged and the supernatant solution was introduced onto the column.

500 mg of dissolved extract (on the column)

Sephadex-LH-20 column: internal diameter: 1.4 cm; length: 27 cm

Open column chromatography: methanol as eluent; 65 test tubes with on average 2.5 ml of solution. Drop rate: between 10 and 17 drops per minute.

Control of the test tubes by DC, conditions: silica gel plates (Merck, silica gel 60 F254), flow agent: EtOAc:HCOOH:CH3COOH:H2O, 100:11:11:27, v:v:v:v; detection: natural substance reagent. The solutions in the test tubes which had a similar flavonoid pattern were combined, the solvent was rotated off and, after freeze-drying, the residue was weighed.

Factions obtained:

Fr. 1: (RG 21-31): 106.2 mg (containing flavonoid according to DC)

Fr. 2: (RG 33-46): 18.3 mg (containing flavonoid according to DC)

Fr. 3: (RG 47-52): 10.03 mg (containing flavonoid according to DC)

Fractions 1-3 as well as mixtures and overlaps thereof provide suitable extracts after drying.

### EXAMPLE 6

### Preparation of a Concentrated Rooibos Extract with a Further Increased Content of Compound I

Further Purification of Fraction 2 from Example 5 by Medium Pressure LC on an RP18 Column.

System: medium pressure (LPLC)

Column: RP 18 silica gel (50×1.2 cm)

Mobile phase: MeOH:H2O (MeOH 30% to 100%)

Flow rate: 0.7 ml/min

Detection: 280 nm

Number of fractions collected: 70 with in each case approx. 3 ml.

After drying, the combined fraction Nos. 43-46 yielded the desired extract.

### EXAMPLE 7

### Formulation of a Combination of Four Plant Extracts for the Prevention and Treatment of Dementia Disorders

Hard gelatine capsules:

1 capsule contains (mg):

| | |
|---|---|
| Green tea extract | 100 |
| Rooibos extract, unfermented | 75 |
| Rooibos extract, fermented | 75 |
| Ginseng extract | 50 |
| Ascorbic acid | 50 |
| Maltodextrin | 20 |
| Silicon dioxide | q.s. |
| Magnesium stearate | q.s. |
| Daily dose for prevention: | 1-4 capsules |
| Daily dose for therapy: | 3-8 capsules |

For the following Examples 8-16, the extracts were sourced as follows:

Extracts

In the context of the present invention, the extracts stated in the following were preferably used. Alternatively, equivalent extracts can be used.

Green tea extract:

Frutarom Switzerland Ltd.

Rütiwiesstrasse 7

CH-8820 Wädenswil

www.frutarom.com

Ginseng extract:

Frutarom Switzerland Ltd.

Rütiwiesstrasse 7

CH-8820 Wädenswil

www.frutarom.com

Rooibos extract (fermented):

Rooibos Ltd.

Rooibos Avenue

Clanwilliam 8135 RSA

South Africa

Curcuma extract:

Plantextrakt GmbH & Co KG

Dutendorfer Str. 5-7

91487 Vestenbergsgreuth

The unfermented rooibos extract according to Example 4 was used as unfermented rooibos extract.

### EXAMPLE 8

### Formulations of a Combination of Four Plant Extracts for the Prevention and Treatment of Dementia Disorders

Hard gelatine capsules:

1 capsule contains (mg):

| | |
|---|---|
| Curcuma extract | 15 |
| Rooibos extract, unfermented* | 100 |
| Rooibos extract, fermented | 100 |
| Ginseng extract | 50 |
| Ascorbic acid | 50 |
| Piperin | 25 |
| Maltodextrin | 20 |
| Silicon dioxide | q.s. |
| Magnesium stearate | q.s. |
| Daily dose for prevention: | 1-4 capsules |
| Daily dose for therapy: | 3-8 capsules |

*(contains 0.4% of the compound according to formula I)

### EXAMPLE 9

### Formulations of a Combination of Three Plant Extracts for the Prevention and Treatment of Dementia Disorders

Hard gelatine capsules:

1 capsule contains (mg):

| | |
|---|---|
| Green tea extract | 150 |
| Rooibos extract, unfermented* | 100 |
| Ginseng extract | 50 |
| Ascorbic acid | 25 |
| Maltodextrin | 50 |
| Silicon dioxide | q.s. |
| Magnesium stearate | q.s. |

*(contains 0.4% by weight of the compound of formula I)
Daily dose for prevention: 1-4 capsules
Daily dose for therapy: 3-8 capsules

### EXAMPLE 10

### Formulations of a Combination of Three Plant Extracts for the Prevention and Treatment of Dementia Disorders

Hard gelatine capsules:

1 capsule contains (mg):

| | |
|---|---|
| Green tea extract | 100 |
| Rooibos extract, unfermented* | 100 |
| Rooibos extract, fermented | 150 |
| Ascorbic acid | 30 |
| Maltodextrin | 0 |
| Silicon dioxide | q.s. |
| Magnesium stearate | q.s. |

*(contains 0.4% by weight of the compound according to formula I)
Daily dose for prevention: 1-4 capsules
Daily dose for therapy: 3-8 capsules

### EXAMPLE 11

### Formulations of a Combination of Three Plant Extracts for the Prevention and Treatment of Dementia Disorders

Hard gelatine capsules:

1 capsule contains (mg):

| | |
|---|---|
| Curcuma extract | 25 |
| Rooibos extract, unfermented* | 100 |
| Rooibos extract, fermented | 200 |
| Ascorbic acid | 30 |
| Maltodextrin | 25 |
| Silicon dioxide | q.s. |
| Magnesium stearate | q.s. |

*(contains 0.8% by weight of the compound according to formula I)
Daily dose for prevention: 1-3 capsules
Daily dose for therapy: 2-6 capsules

### EXAMPLE 12

### Formulations of a Combination of Two Plant Extracts for the Prevention and Treatment of Dementia Disorders

Hard gelatine capsules:

1 capsule contains (mg):

| | |
|---|---|
| Green tea extract | 200 |
| Rooibos extract, unfermented* | 100 |
| Ascorbic acid | 50 |
| Maltodextrin | 30 |
| Silicon dioxide | q.s. |
| Magnesium stearate | q.s. |

*(contains 0.4% by weight of the compound according to formula I)
Daily dose for prevention: 1-3 capsules
Daily dose for therapy: 3-6 capsules

### EXAMPLE 13

### Formulations of a Combination of Two Plant Extracts for the Prevention and Treatment of Dementia Disorders

Hard gelatine capsules:

1 capsule contains (mg):

| | |
|---|---|
| Rooibos extract, unfermented | 150 |
| Rooibos extract, fermented | 200 |
| Ascorbic acid | 30 |
| Maltodextrin | 0 |
| Silicon dioxide | q.s. |
| Magnesium stearate | q.s. |
| Daily dose for prevention: | 1-3 capsules |
| Daily dose for therapy: | 3-6 capsules |

### EXAMPLE 14

### Formulations of a Combination of Two Plant Extracts for the Prevention and Treatment of Dementia Disorders

Hard gelatine capsules:

1 capsule contains (mg):

| | |
|---|---|
| Rooibos extract, unfermented | 150 |
| Green tea extract | 200 |
| Ascorbic acid | 30 |
| Maltodextrin | 0 |
| Silicon dioxide | q.s. |
| Magnesium stearate | q.s. |
| Daily dose for prevention: | 1-4 capsules |
| Daily dose for therapy: | 3-6 capsules |

### EXAMPLE 15

### Formulations of a Combination of Two Plant Extracts for the Prevention and Treatment of Dementia Disorders

Hard gelatine capsules:

1 capsule contains (mg):

| | |
|---|---|
| Rooibos extract, unfermented | 250 |
| Ginseng extract | 75 |
| Ascorbic acid | 30 |
| Maltodextrin | 25 |
| Silicon dioxide | q.s. |
| Magnesium stearate | q.s. |
| Daily dose for prevention: | 1-2 capsules |
| Daily dose for therapy: | 2-4 capsules |

### EXAMPLE 16

### Formulations of a Combination of Two Plant Extracts for the Prevention and Treatment of Dementia Disorders

Hard gelatine capsules:

1 capsule contains (mg):

| | |
|---|---|
| Rooibos extract, unfermented | 250 |
| Curcuma extract | 50 |
| Ascorbic acid | 50 |
| Maltodextrin | 35 |
| Silicon dioxide | q.s. |
| Magnesium stearate | q.s. |
| Daily dose for prevention: | 1-2 capsules |
| Daily dose for therapy: | 2-4 capsules |

### EXAMPLE 17

### Preparation of a Hard Gelatine Capsule

| | |
|---|---|
| Green tea extract | 250 mg |
| Rooibos extract, unfermented | 50 mg |
| Ginseng extract | 50 mg |
| Ascorbic acid | 25 mg |
| Maltodextrin | 25 mg |
| Silicon dioxide and magnesium stearate | q.s. |

### EXAMPLE 18

### Preparation of a Hard Gelatine Capsule

| | |
|---|---|
| Green tea extract (60% Epigalocatechingallate) | 150 mg |
| Rooibos extract, unfermented | 50 mg |
| Ascorbic acid | 25 mg |
| Maltodextrin | 25 mg |
| Silicon dioxide and magnesium stearate | q.s. |

### EXAMPLE 19

### Preparation of a Hard Gelatine Capsule

| | |
|---|---|
| Green tea extract | 100.0 mg |
| Rooibos tea extract | 75.0 mg |
| Green rooibos tea extract* | 75.0 mg |
| Ginseng extract | 50.0 mg |
| Ascorbic acid | 50.0 mg |
| Magnesium stearate (q.s. e.g.: 0.57%) | 2.0 mg |
| Aerosil 200 (q.s. e.g.: 0.14%) | 0.5 mg |

*Content of substance according to formula I: 0.1%.

### EXAMPLE 20

### Preparation of a Lozenge which can also be Dissolved to Produce a Tea Drink (=Lozenge/Tea Pastille)

| Green tea extract | 43 mg |
|---|---|
| Rooibos tea extract | 32 mg |
| Green rooibos tea extract | 32 mg |
| Ginseng extract | 21 mg |
| Ascorbic acid | 22 mg |
| Base | 1,850 mg |
| Base: | % |
| Gum arabic | 29.0 |
| Sugar | 24.0 |
| Glucose syrup | 24.0 |
| Water | 22.6 |
| Flavouring mixture | 0.4 |

*Content of substance according to formula I: 0.18%.

### EXAMPLE 21

### Preparation of a Lozenge which can also be Dissolved to Produce a Tea Drink (=Lozenge/Tea Pastille)

| Green tea extract | 30 mg |
|---|---|
| Green rooibos tea extract | 60 mg |
| Ginseng extract | 20 mg |
| Ascorbic acid | 35 mg |
| Base | 2,015 mg |
| Base: | % |
| Gum arabic | 30.0 |
| Sugar | 12.0 |
| Fructose | 12.0 |
| Apple syrup | 21.0 |
| Water | 24.4 |
| Flavouring mixture | 0.6 |

*Content of substance according to formula I: 0.18%.

### EXAMPLE 22

### Demonstration of Efficacy

A randomised double blind study on people was carried out to verify the improvement in human memory performance by a green rooibos-rooibos-green tea-ginseng capsule (described in Example 19). A placebo capsule containing only microcrystalline cellulose was administered as a comparative preparation. As the dosage regime, 2×3 hard capsules were administered daily before breakfast and before lunch over a period of four weeks.

The test was evaluated by quantitative-topographical EEGs at rest and in the presence of provocations. In addition, the subjects filled out questionnaires.

The present study shows, after a single dose of the capsules with extract of green rooibos, rooibos, green tea and ginseng, lower delta and alpha performance when eyes are closed compared to the placebo. Observation of the frontotemporal brain region which is significant for cognitive functions points to statistically significant differences in the frequency pattern. In particular, when the CPT and the memory test were carried out, at the combined electrode positions F7, T3, Fz, T5 there is an increased fall in the delta and alpha 1 waves under verum conditions.

After a four week repetitive administration of the preparations, it was also possible to show for all of the electrode positions significant differences between placebo and verum during the implementation of CPT and the memory test. Whereas, during the CPT, the alpha and beta waves again decreased more markedly than in the case of the placebo, when the memory test was carried out, in addition to a reduction in the delta waves—also after a single dose—there was a statistically significant reduction in the alpha 1 waves. Since the reduction in the alpha and beta waves during implementation of these tests correlates with the psychometric results and is also generally considered in the literature as a surrogate parameter for memory functions, the result is evaluated as an improvement in the cognitive performance.

The result of the study provides first indications of an improvement in cognitive performance even after taking a single dose of capsules which contain a mixture of extracts of green rooibos, rooibos, green tea and ginseng. In this respect, memory processes in particular seem to be affected. Also after repetitive administration, it was possible to show statistically noticeable differences one hour after administration compared to administration of the placebo, which differences are to be interpreted as a positive influence on memory functions.

1. A combination of plant extracts comprising an extract obtained from unfermented rooibos together with at least two other extracts selected from the group consisting of, fermented rooibos extract, ginseng extract, green tea extract and curcuma extract, wherein said extract obtained from unfermented rooibos comprises at least 0.05% by weight of a compound of formula I 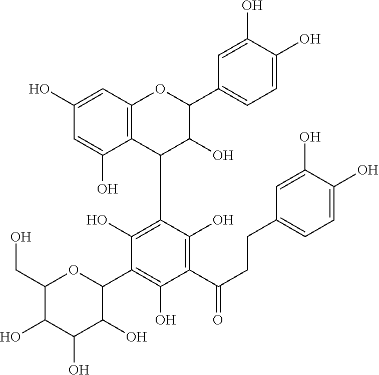
or a pharmaceutically acceptable salt, derivatives or esters thereof.

2. The combination according to claim 1, wherein the extract obtained from unfermented rooibos comprises at least 0.1% by weight of the compound according to formula I or its pharmaceutically acceptable salt, derivative or ester.

3. The combination according to claim 1, wherein the extract obtained from unfermented rooibos comprises at least 0.4% by weight of the compound of formula I or its pharmaceutically acceptable salt, derivative or ester.

4. (canceled)

5. The combination according to claim 1, wherein said combination comprises a dry extract of unfermented rooibos together with at least three further dry extracts selected from the group consisting of dry fermented rooibos extract, dry ginseng extract, dry green tea extract and dry curcuma extract.

6. The combination according to claim 1, wherein said extract of unfermented rooibos is prepared by (a) drying and crushing an unfermented rooibos raw material, (b) extracting the raw material of step (a) with an extracting agent consisting of an alcohol in a quantity of between 20 and 50% (vol./vol.) and water for a predetermined time at a temperature of up to 90° C., and (c) filtering and subsequently concentrating the extract of step (b) to dryness under reduced pressure.

7. The combination according to claim 6, wherein the extract concentrated to dryness is dissolved and further purified by chromatography.

8. The combination according to claim 6, wherein the extraction agent of step (b) consists of ethanol and/or methanol in a quantity of from 20 to 30% by weight and water.

9. The combination according to claim 1, wherein said combination contains a dry extract of green tea prepared by extraction with water or a water-alcohol mixture comprising up to 80% (vol./vol.) alcohol.

10. The combination according to claim 1, wherein said combination contains a dry extract of fermented rooibos prepared by extraction with water or a mixture of water and alcohol.

11. The combination according to claim 1, wherein said combination contains a dry extract of curcuma prepared by extraction with alcohol, CO₂, or a mixture of water with alcohol and/or acetone.

12. The combination according to claim 1, wherein said combination contains a dry extract of ginseng prepared by extraction with a water-alcohol mixture comprising up to 80% (vol./vol.) alcohol.

13. The combination according to claim 1, wherein the unfermented rooibos extract has a total flavenoid content of at least 17% by weight.

14. A drug formulated for the prevention and treatment of dementia disorders comprising a pharmaceutically effective amount of the combination of claim 1.

15. A food supplement comprising the combination of claim 1.

16. (canceled)

17. The combination according to claim 1, wherein said extract of unfermented rooibos is prepared by (a) drying and crushing an unfermented rooibos raw material, (b) extracting the raw material of step (a) with an extracting agent consisting of an alcohol in a quantity of between 20 and 50% (vol./vol.) and water for a predetermined time at a temperature of up to 60° C., and (c) filtering and subsequently concentrating the extract of step (b) to dryness under reduced pressure.

18. The combination according to claim 6, wherein the extract concentrated to dryness is dissolved and further purified by size-exclusion chromatography.
